(19) United States
(12) Patent Application Publication
(10) Pub. No.: US 2024/0207419 A1
(43) Pub. Date: Jun. 27, 2024

(54) SOLID FORM OF 2-CHLORO-2'-DEOXY-ADENOSINE COMPLEX WITH HPßCD

(71) Applicant: BIOPHORE INDIA PHARMACEUTICALS PVT. LTD., Hyderabad (IN)

(72) Inventors: Manik Reddy PULLAGURLA, Hyderabad (IN); Jagadeesh Babu RANGISETTY, Hyderabad (IN)

(21) Appl. No.: 18/288,402

(22) PCT Filed: Apr. 9, 2022
(86) PCT No.: PCT/IB2022/053336

§ 371 Date: Oct. 26, 2023

(30) Foreign Application Priority Data
Apr. 28, 2021 (IN) IN 202141019490

(51) Int. Cl. A61K 47/61

(52) U.S. Cl. CPC A61K 47/61

(57) ABSTRACT

A pure amorphous form of 2-chloro-2′-deoxy-adenosine (Cladribrine (1)) complex with HPβCD is provided, wherein the percentage of crystallinity is less than 0.5% (w/w), or alternatively less than 0.2% (w/w). Also provided is a process for the preparation of amorphous form of Cladribine (1) complex with pharmaceutically suitable excipient having percentage of crystallinity less than 0.5%, or alternatively less than 0.2%. (w/w). Cladribine of formula (1)

### CROSS-REFERENCE TO RELATED APPLICATIONS

This application claims priority to PCT Application No. PCT/IB2022/053336, having a filing date of Apr. 9, 2022, which is based on IN 202141019490, having a filing date of Apr. 28, 2021, the entire contents both of which are hereby incorporated by reference.

### FIELD OF TECHNOLOGY

The following provides pure amorphous form of 2-chloro-2′-deoxy-adenosine complex with pharmaceutical acceptable excipient wherein the percentage of crystallinity is less than 5% (w/w), or alternatively less than 2% (w/w).

The following provides an improved process for the preparation of amorphous form of 2-chloro-2′-deoxy-adenosine (Cladribine (1) complex with HPβCD, wherein the percentage of crystallinity is less than 5% (w/w), or alternatively less than 2% (w/w).

The following provides an improved process for the preparation of amorphous form of 2-chloro-2′-deoxy-adenosine (Cladribine (1) complex with HPβCD. having a purity greater than 99% (w/w) by high performance liquid chromatography (HPLC).

### BACKGROUND

Cladribine is a nucleoside metabolic inhibitor, which is a white or almost white, non-hydroscopic, crystalline powder with the molecular formula C₁₀H₁₂ClN₅O₃ and molecular weight 285.69. It differs in structure from the naturally occurring nucleoside, deoxyadenosine, by the substitution of chlorine for hydrogen in the 2-position of the purine ring. The chemical name of cladribine is 2-chloro-2′-deoxy-adenosine. It is marketed under the trade name MAVENCLAD.

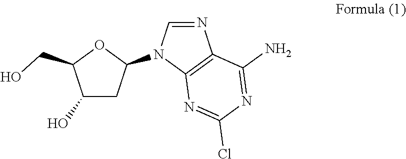 [SMILES: Nc1nc(Cl)nc2c1ncn2[C@H]1C[C@H](O)[C@@H](CO)O1]

The synthesis of Cladribine (1) has been reported in patents and articles, the contents of which are hereby incorporated as reference in their entirety.

U.S. Pat. No. 6,884,880B2 discloses process for the preparation of Cladribine (1), which involves reacting 2-Chloroadenine with heptanoic anhydride to obtain 2-Chloro-6-heptanoylamido purine and converting to its salt form and further reacting with p-toluoyl-α-D-erythropentofuranose derivative to form 6-Chloro-9-(2-deoxy-3,5-di-O-p-toluoyl-β-D-erythropentofuranosyl) purine, which is further reduced to obtain Cladribine.

Though there are conventional art process' for the preparation of Cladribine and intermediate the overall yield obtained according to the process is very less. The Hence, there is a need to develop a process which is less laborious, industrially scalable, economical and which will produce Cladribine in good yield and high purity.

### SUMMARY

An aspect relates to a pure amorphous form of 2-chloro-2′-deoxy-adenosine complex with pharmaceutical acceptable excipient wherein the percentage of crystallinity is less than 5% (w/w), or alternatively less than 2% (w/w).

Embodiments of the present invention provide an improved process for the preparation of amorphous form of 2-chloro-2′-deoxy-adenosine (Cladribine (1) complex with HPBCD, wherein the percentage of crystallinity is less than 5% (w/w), or alternatively less than 2% (w/w).

In one embodiment the present invention provides amorphous Cladribine complex having enhanced dissolution and stability properties.

In one embodiment, the present invention provides a process for the preparation of amorphous form of 2-chloro-2′-deoxy-adenosine complex with HPβCD.

In another embodiment, the present invention provides process for the preparation of complex of Cladribine with pharmaceutically suitable excipient.

In another embodiment, the present invention provides process for the preparation of complex of Cladribine with pharmaceutically suitable excipient selected from the group comprising of lactose, sorbitol, mannitol, saccharose, cellulose, methyl cellulose, ethyl cellulose, microcrystalline cellulose (MCC), polyethylene glycol(PEG), polyvinylpyrrolidone (PVP), polyvinylpyrrolidone K-30, (PVP K-30), polyvinyl acetate hydroxyethyl cellulose (HEC), hydroxy propyl methyl cellulose (HPMC), hydroxypropyl cellulose (HPC), hydroxy propyl methyl cellulose acetate succinate (HPMC-AS), α-cyclodextrin, β-cyclodextrin, γ-cyclodextrin, sulfobutylether-β-cyclodextrin (SBCED) or the like, desirably hydroxypropyl beta cyclodextrin (HPβCD). Embodiments of the present invention provide pure amorphous form of 2-chloro-2′-deoxy-adenosine (1) complex with pharmaceutical acceptable excipient wherein the percentage of crystallinity is less than 5% (w/w), or alternatively less than 2% (w/w).

Embodiments of the present invention provide an improved process for the preparation of amorphous form of 2-chloro-2′-deoxy-adenosine (Cladribine (1) complex with HPβCD, wherein the percentage of crystallinity is less than 5% (w/w), or alternatively less than 2% (w/w).

In one aspect Embodiments of the the present invention provides Cladribine complex having enhanced dissolution and stability properties.

In another aspect Embodiments of the the present invention provide a process for the preparation of amorphous Cladribine complex as illustrated in scheme 1, comprising:
i. providing a mixture of cladribine and suitable pharmaceutically acceptable excipient;
ii. blending or grinding the contents to get the uniform mixture; and
iii. isolating amorphous complex of cladribine with suitable pharmaceutically acceptable excipient.

In another aspect embodiments of the present invention relate to an alternative process for the preparation of amorphous complex of Cladribine with pharmaceutically suitable excipient comprising:
a. dispersing cladribine in a solution containing pharmaceutically acceptable excipient
b. heating the reaction mixture to a suitable temperature
c. filtering the reaction mixture
d. isolating the cladribine complex by using suitable isolation technique.

In another aspect embodiments of the present invention relates to preparation of amorphous Cladribine complex with pharmaceutically suitable excipient with a purity greater than 99% (w/w) by HPLC.

### BRIEF DESCRIPTION

Some of the embodiments will be described in detail, with references to the following Figures, wherein like designations denote like members, wherein:

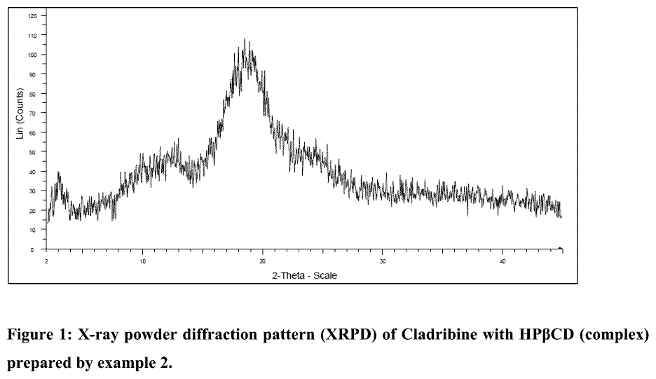

FIG. 1 shows x-ray powder diffraction pattern (XRPD) of Cladribine with HPβCD (complex) prepared by example 2;

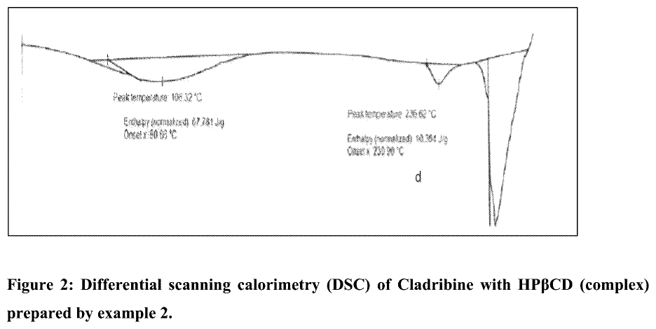

FIG. 2 shows differential scanning calorimetry (DSC) of Cladribine with HPβCD (complex) prepared by example 2;

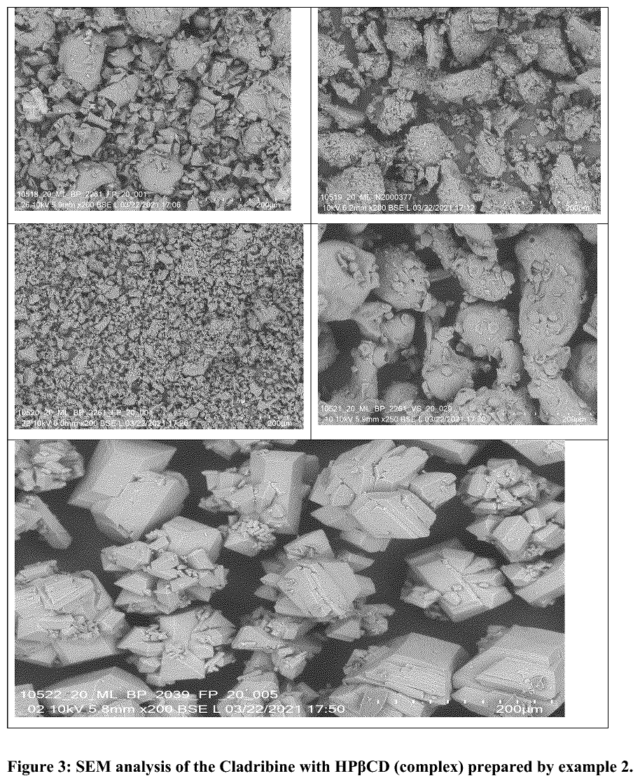

FIG. 3 show SEM analysis of the Cladribine with HPβCD (complex) prepared by example 2;

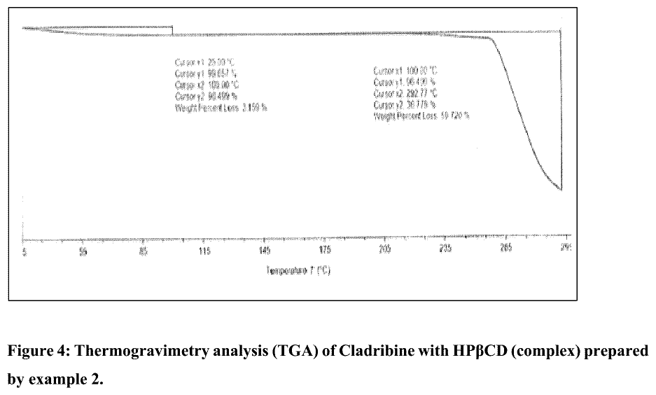

FIG. 4 shows thermogravimetry analysis (TGA) of Cladribine with HPβCD (complex) prepared by example 2;

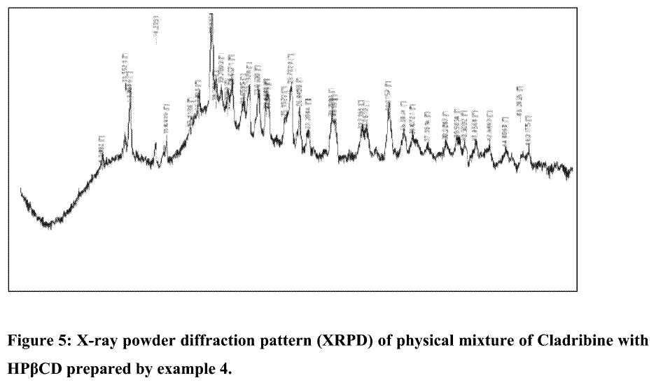

FIG. 5 shows x-ray powder diffraction pattern (XRPD) of physical mixture of Cladribine with HPβCD prepared by example 4; and

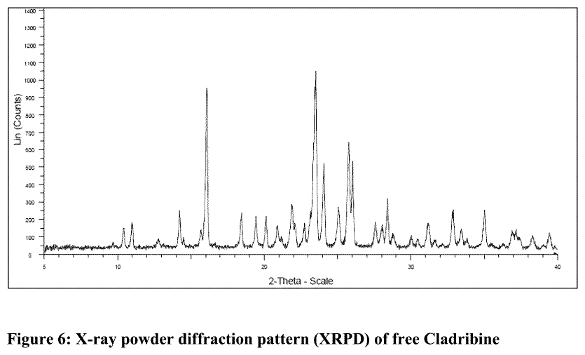

FIG. 6 shows x-ray powder diffraction pattern (XRPD) of free Cladribine.

### DEFINITIONS

The terms used in this specification generally have their ordinary meanings in the conventional art, within the context of embodiments of theinvention and in the specific context where each term is used.

The term “premix” or “premixture” as used herein refers to a pharmaceutical formulation that does not require reconstitution or dilution prior to administration to a patient.

The term “pharmaceutically acceptable.” when used in connection with the pharmaceutical compositions of embodiments of the invention, refers to molecular entities and compositions that are physiologically tolerable and do not typically produce untoward reactions when administered to a human.

The term “excipient” as used herein refers to a non-pharmaceutically active additives used in the manufacture of a pharmaceutical composition, which additive allows the pharmaceutically active ingredient or medicament to be manufactured into a pharmaceutical formulation The term “active ingredient”, as used herein refers to any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect.

The term “sweetener”, as used herein refers to the low-molecular-weight polyhydroxy compounds, but not limited to sucrose, sorbitol, and mannitol, but various other groups may give intensely sweet compounds. Imides such as saccharin, amino acid combinations such as those in aspartame, and some chlorinated sugars such as sucralose are very sweet

The term “lubricants”, as used herein refers to an additive to reduce friction, is an essential component of a drug formula. Pharmaceutical lubricants are the agents added to tablet and capsule formulations in a very small quantity (usually 0.25%-5.0%, w/w) to improve the powder processing properties of formulations.

Unless defined otherwise, all technical and scientific terms used herein have the same meanings as commonly understood by one skilled in the conventional art.

### DETAILED DESCRIPTION

Embodiments of the present invention provide pure amorphous form of 2-chloro-2′-deoxy-adenosine complex with pharmaceutical acceptable excipient wherein the percentage of crystallinity is less than 5% (w/w), or alternatively less than 2% (w/w).

Embodiments of the present invention provide an improved process for the preparation of amorphous form of 2-chloro-2′-deoxy-adenosine (Cladribine (1) complex with HPβCD, wherein the percentage of crystallinity is less than 5% (w/w), or alternatively less than 2% (w/w).

In one embodiment the present invention provides amorphous Cladribine complex having enhanced dissolution and stability properties.

More specifically, embodiments of the present invention provide pure solid form of cladribine complex that is at least about 70% pure (i.e., free of any crystalline forms), at least about 80% pure, at least about 90% pure, at least about 95% pure, at least about 99% pure, and/or at least about 99.9% pure.

In another embodiment, the present inventors found that solid form of Cladribine complex exists in a pure amorphous form or in a combination of amorphous and crystalline forms. Cladribine (1) complex can be prepared by mixing Cladribine (1) with HPβCD in a suitable solvent. The solid form of Cladribine (1) complex with suitable excipient can be prepared by Lyophilization, distillation or by partial distillation or slow evaporation or slow cooling. Cladribine (1) complex with HPβCD prepared by distillation is industrially suitable method and the most economically feasible method. Hence the solid form of Cladribine (1) complex prepared by distillation method analyzed by X-ray diffraction methods. Further, embodiments of the present invention provide the method to prepare pure solid form of Cladribine (1) premix, wherein the percentage of crystallinity is identified less than 0.5% (w/w) and or alternatively less than 0.2% (w/w).

In another embodiment, the present invention relates to an improved process for the preparation of amorphous Cladribine complex with a purity greater than 99% by HPLC as illustrated in scheme 1.

In another embodiment of the invention the present invention provides Cladribine complex having enhanced stability and dissolution properties that can be easily formulated into pharmaceutical compositions.

In another embodiment, the present invention provides process for the preparation of complex without using any solvent or simply by blending of the contents at a suitable temperature.

In another embodiment, the present invention provides a process for preparation of amorphous Cladribine (1) complex comprising the steps of:
i. providing a mixture of cladribine and suitable pharmaceutically acceptable excipient.
ii. Blending or grinding the contents to get the uniform mixture; and
iii. Isolating amorphous complex of cladribine with suitable pharmaceutically acceptable excipient.

Cladribine (1) complex with HPβCD obtained by blending of Cladribine (1) with suitable excipient desirably HPβCD contains more than 5% of crystalline nature as shown in FIG. 5.

In yet another embodiment, the present invention relates to an alternative process for the preparation of amorphous complex of Cladribine with pharmaceutically suitable excipient desirably with HPβCD Complex, which comprises.
a. dispersing cladribine in a solution containing pharmaceutically acceptable excipient
b. heating the reaction mixture to a suitable temperature
c. filtering the reaction mixture
d. isolating the cladribine complex by using suitable isolation technique.

The process involves dispersing Cladribine with hydroxypropyl betadex (HPβCD) dissolved in suitable solvent and heated to temperature at 30-70° C., or at 40-50° C., cooled and dried to obtain Cladribine: HPβCD Complex.

Pharmaceutically suitable excipient used in embodiments of the present invention as dispersnig agent is selected from the group comprising of lactose, sorbitol, mannitol, saccharose, cellulose, methyl cellulose, ethyl cellulose, microcrystalline cellulose (MCC), polyethylene glycol(PEG), polyvinylpyrrolidone (PVP), polyvinylpyrrolidone K-30, (PVP K-30), polyvinyl acetate hydroxyethyl cellulose (HEC), hydroxy propyl methyl cellulose (HPMC), hydroxypropyl cellulose (HPC), hydroxy propyl methyl cellulose acetate succinate (HPMC-AS), α-cyclodextrin, β-cyclodextrin, γ-cyclodextrin, sulfobutylether-β-cyclodextrin (SBCED) or the like, desirably Hydroxypropyl Betadex (HPβCD).

In another embodiment, the solvent used in the present invention is selected from the group comprising of methanol, ethanol, water or mixtures thereof.

The cladribine used in embodiments of the present invention is selected from crystalline form of amorphous form of Cladribine free base or pharmaceutically acceptable salts.

In another embodiment, the solvent is removed by evaporation. Evaporation can be achieved by lyophilization or freeze drying, or spray drying or by distillation at a suitable temperature.

In another embodiment, the distillation can be performed at atmospheric pressure or at reduced pressure.

In another embodiment, amorphous cladribine (1) complex obtained by distillation method is having percentage of crystallinity less than 5.0% (w/w) and desirably less than 2.0% (w/w) as shown in FIG. 6.

In another embodiment, pharmaceutically acceptable excipients may be utilized as required for conversion of the premixes into the final pharmaceutical dosage forms. Which include any one or more diluents, binders, stabilizers, lubricants, surfactants or other additives commonly used in solid pharmaceutical dosage form preparations.

In another embodiment, Comparative study conducted to evaluate the dissolution profile of cladribine complex with HPβCD prepared by distillation is illustrated as shown in table 1. The dissolution parameters are as shown below.
A. Medium: pH 6.8 Phosphate Buffer
B. Apparatus: USP Type II apparatus
C. RPM: 50 rpm
D. Media volume: 900 ml
E. Time points for sample collection (in minutes): 5, 10, 15, 20, 30 and infinity

TABLE 1
Dissolution profile of Cladribine (1)
complex prepared in different methods.

| | | | |
|---|---|---|---|
| Dissolution Time | Batch 01 | Batch 02 | Batch 03 |

| | | | | |
|---|---|---|---|---|
| 5 | min | 84 | 93 | 93 |
| 10 | min | 97 | 103 | 103 |
| 15 | min | 98 | 103 | 103 |
| 20 | min | 98 | 102 | 102 |
| 30 | min | 98 | 103 | 103 |

| | | | |
|---|---|---|---|
| Infinity | 98 | 103 | 103 |

The details of batches used in the dissolution study is as follows. Batch no-01 is prepared by distillation and Batch 02 is partially distilled and batch no-03 is partially distilled and micronized.

In another embodiment, amorphous Cladribine complex obtained according to the present invention is characterised by X-ray powder diffractions (XRD) pattern and DSC thermogram as illustrated in FIG. 1 and FIG. 2.

In yet another embodiment, Cladribine with pharmaceutically suitable excipient is having a weight ratio of 1:10 to 1:16.

In yet another embodiment, Cladribine: HPβCD complex obtained according to the present invention is having percentage of crystallinity less than 5% (w/w) at the end the distillation. This is further supported by absence of crystalline peaks from X-ray diffraction method.

In yet another embodiment, Cladribine: HPβCD Complex obtained according to the present invention has moisture content (MC) less than 10.0%.

In yet another embodiment, amorphous Cladribine (1) complex obtained according to the present invention is having purity greater than 99% by HPLC.

In yet another embodiment, Cladribine obtained according to the present invention has methanol content and ethanol content less than 1200 ppm, or less than 500 ppm.

In yet another embodiment, Cladribine complex obtained according to the present invention has moisture content (MC) less than 4.0%, or less than 2.0%.

The following examples further illustrate embodiments of the present invention but should not be construed in any way as to limit its scope.

### EXAMPLES

### Example 1: Preparation of Cladribine (1) Premix

10 gm of Cladribine is added to 144 gm of HPβCD dissolved in 700 L of water at a temperature of 40-50° C. The reaction mass was stirred at 40-50° C. for 8-12 hrs, cooled to 25-30° C. The reaction mass was further stirred for 6-8 hrs then passed through micron filter. The filtrate was distilled off completely under vacuum to obtain Cladribine: HPβCD Complex. Purity: 99.86% (w/w).

### Example 2: Preparation of Cladribine (1) Premix

10 gm of Cladribine is added to 144 gm of HPβCD dissolved in 700 L of water at a temperature of 40-50° C. The reaction mass was stirred at 40-50° C. for 8-12 hrs, cooled to 25-30° C. The reaction mass was further stirred for 6-8 hrs then passed through micron filter. The filtrate was lyophilized to obtain Cladribine: HPβCD Complex. Purity: 99.86% (w/w).; XRD: FIG. 1, DSC: FIG. 2, SEM: FIG. 3.

### TGA: FIG. 4.

### Example 3: Preparation of Amorphous Cladribine Premix

10 gms of Cladribine is added to 144 gm of HPβCD dissolved in 700 L of water at a temperature of 40-50° C. The reaction mass was stirred at 40-50° C. for 8-12 hrs, cooled to 25-30° C. The reaction mass was further stirred for 6-8 hrs then passed through micron filter. The filtrate was partially distilled off under vacuum and dried the solvent in tray drier to obtain Cladribine: HPβCD Complex. Purity: 99.86% (w/w).

### Example 4: Preparation of Amorphous Cladribine Complex

10 gms of Cladribine is added to 144 gm of HPβCD. The reaction mixture is bended until the uniform mixture is obtained. Purity: 99.86% (w/w); XRD: FIG. 5.

Although the invention has been illustrated and described in greater detail with reference to the exemplary embodiments, the invention is not limited to the examples disclosed, and further variations can be inferred by a person skilled in the conventional art, without departing from the scope of protection of the invention.

For the sake of clarity, it is to be understood that the use of “a” or “an” throughout this application does not exclude a plurality, and “comprising” does not exclude other steps or elements.

We claim:

1. An amorphous Cladribine complex of Formula (1) with at least one pharmaceutically acceptable excipient. 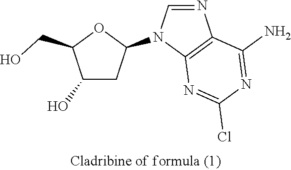 [SMILES: Nc1nc(Cl)nc2c1ncn2[C@H]1C[C@H](O)[C@@H](CO)O1]
wherein the amorphous cladribine complex having percentage of crystallinity less than 2% (w/w).

2. A process for the preparation of stable pharmaceutical composition of amorphous Cladribine complex of Formula (1) comprises:
a) providing a solution of Cladribine in a suitable pharmaceutically acceptable excipient as dispersing agent,
b) removing the solvent from the solution obtained in step a); and
c) recovering amorphous form of Cladribine complex of formula (1).

3. A process for the preparation of stable pharmaceutical composition of amorphous Cladribine complex of Formula (1) comprises:
a) mixing Cladribine with a suitable dispersing agent or pharmaceutically acceptable salt;
b) blending or grinding the mixture to obtain amorphous Cladribine complex with pharmaceutically suitable agent; and
wherein the amorphous cladribine complex is having % of crystallinity less than 2% (w/w).

4. The process of claim 2, wherein suitable solvent in step a) is selected from alcohols, esters, ketones, hydrocarbons, water, or mixtures thereof.

5. The process of claim 2, wherein removal of solvent in step b) is affected by evaporation, freeze drying, spray drying, lyophilization, by addition of suitable anti-solvent or any combination thereof.

6. The process of claim 2, wherein step c) involves an additional step of drying the isolated Cladribine with suitable dispersing agent.

7. A solid dispersion comprising amorphous Cladribine in a dispersing agent.

8. The solid dispersion of claim 7, wherein the dispersing agent comprises HPBCD, hydroxypropyl methyl cellulose (HPMC), Polyvinyl pyrrolidone (PVP), Colloidal silicon dioxide and the like.

9. A pharmaceutical composition comprising solid dispersion of claim 7.
